(19) United States
(12) Patent Application Publication
(10) Pub. No.: US 2026/0317798 A1
(43) Pub. Date: Oct. 15, 2026

(54) METHOD FOR THE GENERATION OF SINGLE STRANDED DNA

(71) Applicant: Hoffmann-La Roche Inc., Little Falls, NJ (US)

(72) Inventors: Fiona KAPPAUN, Munich (DE); Julia MANZ, Penzberg (DE); Johannes Anton PSCHIRER, München (DE); Raphael RUPPERT, Wielenbach (DE)

(73) Assignee: Hoffmann-La Roche Inc., Little Falls, NJ (US)

(21) Appl. No.: 19/548,109

(22) Filed: Feb. 24, 2026

Related U.S. Application Data
(63) Continuation of application No. PCT/EP2024/073796, filed on Aug. 26, 2024.

(30) Foreign Application Priority Data
Aug. 29, 2023 (EP) 23193960.4

(51) Int. Cl. C12P 19/34; C12N 15/86

(52) U.S. Cl. CPC C12P 19/34; C12N 15/86; C12N 2750/14143; C12N 2750/14151

(57) ABSTRACT

The present invention relates to a method for the generation of single stranded DNA (ssDNA) molecules from circular double stranded DNA (dsDNA) molecules.

### CROSS REFERENCE TO RELATED APPLICATIONS

This application is a Continuation of International Application No. PCT/EP2024/073796, filed Aug. 26, 2024, claims priority to European Patent Application No. 23193960.4 filed Aug. 29, 2023, each of which is incorporated herein by reference in its entirety.

### FIELD OF THE INVENTION

The present invention relates to the generation of single stranded DNA (ssDNA) starting from double stranded DNA (dsDNA).

### BACKGROUND

Gene therapy aims to cure or even prevent diseases by delivering the appropriate genetic information. Adeno-associated viruses (AAV) based vectors (have been successfully used as vehicles for the therapeutic nucleic acids with currently two AAV-based drugs ap-proved in the US and the EU (as of June 2022). To ensure the efficacy and safety of the therapy, it is essential to control the quality of the active pharmaceutical ingredient, i.e., the nucleic acids. Single stranded AAV DNA is needed as homogenous standard for the development of analytical methods to determine the quality of the produced AAV particles. Additionally, ssDNA could be used for in vitro production of AAV particles. Therefore, there is a need for a method for the generation of ssDNA.

### SUMMARY

In a first aspect, the present invention provides a method for the generation of single stranded DNA (ssDNA) molecules from circular double stranded DNA (dsDNA) molecules comprising the following steps:
a) providing circular dsDNA molecules,
b) cutting the circular dsDNA molecules by a first restriction enzyme resulting in linear dsDNA molecules,
c) enzymatic dephosphorylation of the linear dsDNA molecules,
d) cutting the linear dsDNA molecules by at least a second restriction enzyme,
e) incubate dsDNA with an exonuclease having specificity for DNA strands with a 5′ phosphate end thereby generating ssDNA molecules.

In an embodiment of the method of the present invention, in step d) the linear ds DNA molecules are cut by two restriction enzymes or a restriction enzyme recognizing two sites in the dsDNA molecules.

In an embodiment of the method of the present invention, the ds DNA in step a) is an AAV plasmid comprising a human gene construct and a plasmid backbone fragment.

In an embodiment of the method of the present invention, the restriction enzyme in step d) generates an AAV plasmid backbone fragment and a human gene construct fragment.

In an embodiment of the method of the present invention, the restriction enzyme in step b) recognizes a cleavage site in the AAV plasmid backbone fragment.

In an embodiment of the method of the present invention, the restriction enzyme in step b) recognizes a cleavage site at the interface of the AAV plasmid backbone fragment and the human gene construct fragment.

In an embodiment of the method of the present invention, the enzymatic dephosphorylation in step c) is done by a phosphatase.

In a second aspect, the present invention provides the use of the ssDNA produced according to the method of the present invention for the in vitro production of AAV viral particles.

### SHORT DESCRIPTION OF THE FIGURES

FIG. 1 shows the workflow of the method of the present invention with the use of two restriction enzymes.

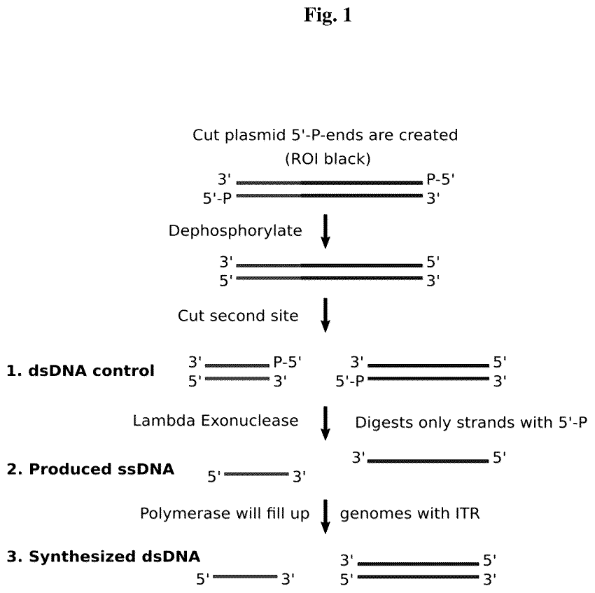

FIG. 2 shows the workflow of the method of the present invention with the use of three restriction enzymes.

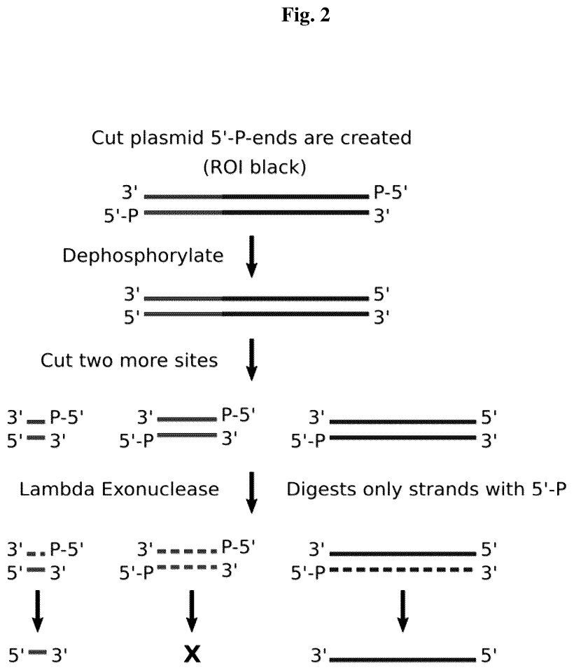

FIG. 3 shows an agarose gel with the DNA bands generated by the ssDNA generating method using the restriction enzymes PacI and StuI and the fill-up of the resulting ssDNA to dsDNA using a DNA-polymerase.

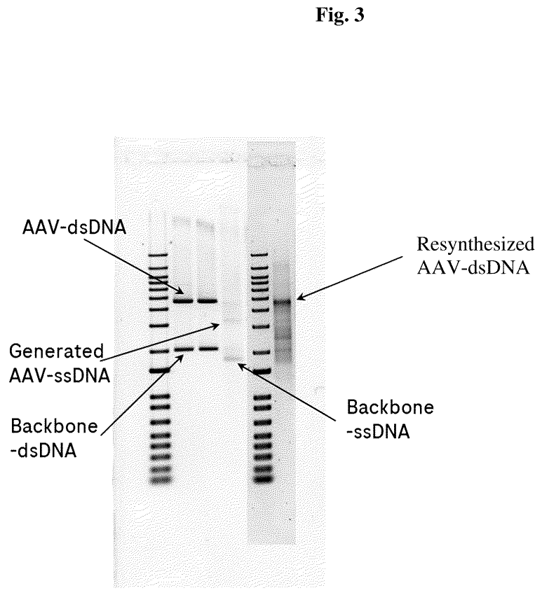

### DETAILED DESCRIPTION OF EMBODIMENTS OF THE INVENTION

Useful methods and techniques for carrying out the subject matter of the current invention are described in e.g. Ausubel, F.M. (ed.), Current Protocols in Molecular Biology, Volumes I to III (1997); Glover, N.D., and Hames, B.D., ed., DNA Cloning: A Practical Approach, Volumes I and II (1985), Oxford University Press; Freshney, R.I. (ed.), Animal Cell Culture-a practical approach, IRL Press Limited (1986); Watson, J.D., et al., Recombinant DNA, Second Edition, CHSL Press (1992); Winnacker, E.L., From Genes to Clones; N.Y., VCH Publishers (1987); Celis, J., ed., Cell Biology, Second Edition, Academic Press (1998); Freshney, R.I., Culture of Animal Cells: A Manual of Basic Technique, second edition, Alan R. Liss, Inc., N.Y. (1987). The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed muta-genesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B.D., and Higgins, S.G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The terms “comprise(s)”, “include(s)”, “having”, “has”, “can”, “contain(s)” and variants thereof, as used herein, are intended to be open-ended transitional phrases, terms or words that do not preclude the possibility of additional acts or structures. The term “comprising” also encompasses the term “consisting of”. The current invention also contemplates other embodiments “comprising”, “consisting of” and “consisting essentially of” the embodiments or elements presented herein, whether explicitly set forth or not.

The term “recombinant cell” as used herein denotes a cell after genetic modification, such as, e.g., a cell expressing a heterologous polypeptide of interest and that can be used for the production of said heterologous polypeptide of interest at any scale. For example, “a recombinant mammalian cell comprising an exogenous nucleotide sequence” denotes a cell wherein the coding sequences for a heterologous polypeptide of interest have been introduced into the genome of the host cell. For example, “a recombinant mammalian cell comprising an exogenous nucleotide sequence” that has been subjected to recombinase mediated cassette exchange (RMCE) whereby the coding sequences for a polypeptide of interest have been introduced into the genome of the host cell is a “recombinant cell”.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The terms “vector” or “plasmid”, which can be used interchangeably, as used herein, re-fer to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

The term “nucleic acid molecule” or “polynucleotide” includes any compound and/or substance that comprises a polymer of nucleotides. Each nucleotide is composed of a base, specifically a purine-or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribo-nucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler al, Nature Medicine 2017, published online 12 Jun. 2017, doi: 10.1038/nm.4356 or EP 2 101 823 B1).

The term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed. ; and Rose, Comprehensive Virology 3:1-61(1974)).

An “AAV vector” as used herein refers to a vector comprising one or more polynucleotides of interest (or transgenes) that are flanked by AAV terminal repeat sequences (ITRs).

Such AAV vectors can be replicated and packaged into infectious viral particles when present in a host cell that has been transfected with a vector encoding and expressing rep and cap gene products.

An “AAV viral particle” refers to a viral particle composed of at least one AAV capsid protein and an encapsidated polynucleotide AAV vector. If the particle comprises a heterologous polynucleotide (i.e. a polynucleotide such as a transgene to be delivered to a mammalian cell), it is typically referred to as an “AAV vector particle” or simply an “AAV vector”.

### EXAMPLES

### ssDNA from Plasmids Using Exonuclease

Used plasmid mGL-Kanamycin-resistance (Roche ID: D1AK5211)
1. Digest Plasmid with Agel (NEB)
1 μl (20 u) Agel-HF/μg plasmid DNA
1× rcutSmart
Fill up with Nuclease free water to 50 μl/μg plasmid DNA
Digest: 15 min@37 °C
Heat inactivation: 20 min@65 °C
2. Dephosphorylation (NEB)
Add 1 μl (5 u) Quick CIP/μg plasmid DNA
Dephosphorylation: 10 min@37 °C
Heat inactivation: 2 min@80 °C
3. Digest with PaqCI (NEB)
Add 1 μl (10 u) PaqCI/Mg plasmid DNA
Add 1 μl PaqCI Activator/μg plasmid DNA
Digest: 60 min@37 °C
Heat inactivation: 20 min@65 °C
4. Digest 5′ phosphorylated DNA with Lambda Exonuclease (Thermo)
Add 0.8 μl (8 u) Lambda Exonuclease/μg Plasmid DNA
Add Lambda Exonuclease Reaction Buffer to 1×
Digest: 30 min@37 °C
Heat inactivation: 15 min@80 °C

1. A method for the generation of single stranded DNA (ssDNA) molecules from circular double stranded DNA (dsDNA) molecules comprising the following steps:
a) providing circular dsDNA molecules,
b) cutting the circular dsDNA molecules by a first restriction enzyme resulting in linear dsDNA molecules,
c) enzymatic dephosphorylation of the linear dsDNA molecules,
d) cutting the linear dsDNA molecules by at least a second restriction enzyme,
e) incubate dsDNA with an exonuclease having specificity for DNA strands with a 5′ phosphate end thereby generating ssDNA molecules.

2. The method of claim 1, wherein in step d) the linear ds DNA molecules are cut by two restriction enzymes or a restriction enzyme recognizing two sites in the dsDNA molecules.

3. The method of claim wherein the ds-DNA in step a) is an AAV plasmid comprising a human gene construct and a plasmid backbone fragment.

4. The method of claims 3, wherein the restriction enzyme in step d) generates an AAV plasmid backbone fragment and a human gene construct fragment.

5. The method of claim 3, wherein the restriction enzyme in step b) recognizes a cleavage site in the AAV plasmid backbone fragment.

6. The method of claim 3, wherein the restriction enzyme in step b) recognizes a cleavage site at the interface of the AAV plasmid backbone fragment and the human gene construct fragment.

7. The method of claim 1, wherein the enzymatic dephosphorylation in step c) is done by a phosphatase.

8. Use of the ssDNA produced according to claim 1 for the in vitro production of AAV particles.
